(19) United States
(12) Patent Application Publication
(10) Pub. No.: US 2025/0002847 A1
(43) Pub. Date: Jan. 2, 2025

(54) ENGINEERED EXRACELLULAR VESICLES WITH TAILORED TROPISM

(71) Applicant: OHIO STATE INNOVATION FOUNDATION, Columbus, OH (US)

(72) Inventors: Natalia Higuita-Castro, Columbus, OH (US); Daniel Gallego-Perez, Columbus, OH (US); Ana Salazar-Puerta, Columbus, OH (US)

(21) Appl. No.: 18/700,936

(22) PCT Filed: Oct. 17, 2022
(86) PCT No.: PCT/US2022/078211

§ 371 Date: Apr. 12, 2024

Related U.S. Application Data
(60) Provisional application No. 63/256,127, filed on Oct. 15, 2021.

(51) Int. Cl. C12N 5/079; C12N 15/88

(52) U.S. Cl. CPC C12N 5/0622; C12N 15/88; C12N 2501/60; C12N 2506/1307

(57) ABSTRACT

Disclosed herein are methods for engineering extracellular vesicles (EVs) with a desired cell tropism for a target cell. This method involves reprogramming somatic cells towards the lineage of the target cell and collecting EVs produced by these reprogrammed cells, which will have the desired cell tropism. These EVs can in some embodiments be used to deliver diagnostic and/or therapeutic cargo to the target cells in a subject in need thereof. Therefore, also disclosed herein are methods of treating a disease or condition in a subject that involves engineering the reprogrammed cells to produce therapeutic EVs that deliver a therapeutic agent to the target cells.

### CROSS-REFERENCE TO RELATED APPLICATIONS

This application claims benefit of U.S. Provisional Application No. 63/256, 127, filed Oct. 15, 2021, which is hereby incorporated herein by reference in its entirety.

### BACKGROUND

Exosomes are naturally secreted nanovesicles that have recently aroused a great interest in the scientific and clinical community for their roles in intercellular communication in almost all physiological and pathological processes. These 30-100 nm sized vesicles are released from the cells into the extracellular space and ultimately into biofluids in a tightly regulated way. Their molecular composition reflects their cells of origin, may confer specific cell or tissue tropism and underlines their biological activity. Exosomes and other extracellular vesicles (EVs) carry specific sets of proteins, nucleic acids (DNA, mRNA and regulatory RNAs), lipids and metabolites that represent an appealing source of novel noninvasive markers through biofluid biopsies. Exosome-shuttled molecules maintain their biological activity and are capable of modulating and reprogramming recipient cells. This multi-faceted nature of exosomes hold great promise for improving cancer treatment featuring them as novel diagnostic sensors as well as therapeutic effectors and drug delivery vectors. Natural biological activity including the therapeutic payload and targeting behavior of EVs can be tuned via genetic and chemical engineering. However, it is time consuming to identify and test suitable cell targeting ligands. Improved methods are needed to design EVs with desired cell tropism.

### SUMMARY

Disclosed herein are methods for engineering extracellular vesicles (EVs) with a desired cell tropism for a target cell. This method involves reprogramming somatic cells towards the lineage of the target cell and collecting EVs produced by these “reprogrammed cells,” which will have the desired cell tropism.

In some embodiments, reprogramming somatic cells involves delivering intracellularly into the somatic cells a polynucleotide comprising nucleic acid sequences encoding reprogramming transcription factors (TFs). Examples of reprogramming TFs are provided in Table 1.

In some embodiments, EVs are collected from the reprogrammed cells from 12 hours to 5 days after the reprogramming transcription factors are delivered to the somatic cells, including 12, 24, 36, or 48 hours, and including 1, 2, 3, 4, or 5 days after the reprogramming transcription factors are delivered to the somatic cells. Therefore, in some embodiments, the “reprogrammed cells” are reprogrammed towards the target lineage but are not fully differentiated. In some embodiments, the “reprogrammed cells” express cell markers indicative of progenitor cells for the target cell lineage.

In some embodiments, the reprogrammed cells express lineage specification markers, but lack one or more markers of full lineage differentiation.

These EVs can in some embodiments be used to deliver diagnostic and/or therapeutic cargo to the target cells in a subject in need thereof. Therefore, also disclosed herein are methods of treating a disease or condition in a subject that involves engineering the reprogrammed cells to produce therapeutic EVs that deliver a therapeutic agent to the target cells.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates designer EVs with enhanced tropism for SCs made ex vivo and deployed in vivo to treat neurofibromas.

FIGS. 2A and 2B show how SC-derived EVs are preferentially internalized by SCs compared to fibroblasts. Fibroblast-derived EVs, on the other hand, show enhanced tropism for fibroblasts. FIG. 2C shows SC-derived EVs can be loaded with large plasmid copies (˜10 kb) and transcripts. FIG. 2D shows successful delivery to SCs with designer EVs. *p<0.05.

FIG. 3A shows SC-derived EVs preferentially accumulated in the sciatic nerve (post-crush injury) of mice after systemic delivery. FIG. 3B shows high magnification image of dashed square in FIG. 3A showing labeled EVs. FIG. 3C shows that fibroblast-derived EVs did not show accumulation in the sciatic nerve post-crush.

FIGS. 4A and 4B show how myoblasts derived EVs are preferentially internalized by myoblasts compared to fibroblasts. Fibroblast-derived EVs, on the other hand, show enhanced tropism for fibroblasts *p<0.05.

FIG. 4C shows myoblasts derived EVs preferentially accumulated in the muscle tissue (e.g., gastrocnemius) of mice after systemic delivery. White arrows pointing at the co-localization of labeled EVs (purple) with the green fluorescence protein (GFP) cargo inside the EVs in the tissue, adjacent to the multiple nuclei of muscle cells, counter stained with DAPI (blue). FIG. 4D shows that fibroblast-derived EVs did not show accumulation in the muscle tissue (e.g., gastrocnemius) after systemic EV delivery.

FIG. 5A shows N2A neurons effectively internalize EVs derived from fibroblasts primed to reprogram to neurons (˜100% uptake efficiency). FIG. 5B shows N2A neurons show higher affinity for EVs derived from reprogramming fibroblasts compared to control EVs. *p<0.05.

FIGS. 6A and 6B shows how engineered EVs collected 24 hours after transfection of fibroblasts with the reprograming factors SOX10 alone or the combination of SOX10+EGR2, which induce direct cell reprogramming towards SCs, are preferentially internalized by SCs compared to fibroblasts 6 hours after EV treatment *p<0.05.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W. H. Freeman and Company, New York (1993); Posttranslational Covalent Modification of Proteins, B. C. Johnson, Ed., Academic Press, New York, pp. 1-12 (1983)).

As used herein, the term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

A “nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more nucleotides linked together. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but are linked together through a moiety other than a phosphate moiety. Nucleotide substitutes are able to conform to a double helix type structure when interacting with the appropriate target nucleic acid.

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:
100 times the fraction W/Z,
   where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. By “specifically hybridizes” is meant that a probe, primer, or oligonucleotide recognizes and physically interacts (that is, base-pairs) with a substantially complementary nucleic acid (for example, a c-met nucleic acid) under high stringency conditions, and does not substantially base pair with other nucleic acids.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5× SSC (150 mM NaCl, 15 mM trisodium citrate), 50 mM sodium phosphate (pH 7.6), 5× Denhardt's solution, 10% dextran sulfate, and 20 μg/ml denatured, sheared carrier DNA such as salmon sperm DNA, followed by washing the hybridization support in 0.1× SSC at approximately 65° C. Other hybridization and wash conditions are well known and are exemplified in Sambrook et al, Molecular Cloning: A Laboratory Manual, Second Edition, Cold Spring Harbor, N.Y. (1989), particularly chapter 11.

The “control elements” or “regulatory sequences” are those non-translated regions of the vector—enhancers, promoters, 5′ and 3′ untranslated regions—which interact with host cellular proteins to carry out transcription and translation. Such elements may vary in their strength and specificity.

A “promoter” is generally a sequence or sequences of DNA that function when in a relatively fixed location in regard to the transcription start site. A “promoter” contains core elements required for basic interaction of RNA polymerase and transcription factors and can contain upstream elements and response elements.

“Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers, like promoters, also often contain response elements that mediate the regulation of transcription. Enhancers often determine the regulation of expression.

An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

### Engineered Extracellular Vehicles (EVs) With Designer Tropism

Disclosed herein are methods for engineering extracellular vesicles (EVs) with a desired cell tropism for a target cell. This method involves reprogramming somatic cells towards the lineage of the target cell and collecting EVs produced by these reprogrammed cells, which will have the desired cell tropism.

For example, methods for designer EVs that modulate pro-neuronal cell responses are described in Ortega-Pineda, et al. Adv. Healthcare Mater. 2022 11: 210085, which is incorporated by reference in its entirety for the teaching of these EVs and heir methods of production and use.

### Reprogramming Somatic Cells

Disclosed are methods of reprogramming somatic cells into target cells that involve delivering intracellularly into the somatic cells a polynucleotide comprising nucleic acid sequences encoding reprogramming transcription factors. In some embodiments, the nucleic acid sequences are present in non-viral vectors. In some embodiments, the nucleic acid sequences are operably linked to an expression control sequence. In other embodiments the nucleic acids are operably linked to two or more expression control sequences.

In some embodiments, the donor cells can be any donor cell able to produce EVs, including (but not limited to) skin cells (e.g., fibroblasts, keratinocytes, skin stem cells), adipocytes, dendritic cells, peripheral blood mononuclear cells (PBMC), pancreatic cells (e.g., ductal epithelial cells), liver cells (e.g., hepatocytes), immune cells (e.g., T cells, macrophages, myeloid derived suppressor cells).

Transcription factors that can reprogram somatic cells into target cell types are known in the art. The diversity of cell types found in the adult organism are created in the course of development by lineage-specific transcription factors (TFs) that define and reinforce specific gene expression patterns of each cell type. Fully differentiated cells can be stimulated to go through remarkable cell fate changes by the induction of pluripotency in primary fibroblasts via the combination of four TFs (Yamanaka's TFs) (Vierbuchen T, et al. Biotechnol. 2011 29 (10): 892-907). These transformations have been understood as a reversion of mature into more primitive developmental states, with an associated deletion of developmentally important epigenetic information (Vierbuchen T, et al. Nature. 2010 463 (7284): 1035-41). Nevertheless, direct lineage reprogramming has been considered an attractive approach in regenerative medicine since it provides an alternative method with a relatively lower risk of tumorigenesis when compared to the use of pluripotent stem cells (Ruzittu S, et al. Direct Lineage Reprogramming: Harnessing Cell Plasticity between Liver and Pancreas. Cold Spring Harbor perspectives in biology. 2019; Xu J, et al. Cell Stem Cell. 2015 16 (2): 119-34).

The direct lineage reprogramming can induce cell fate conversion of a functional cell type from one lineage into another without passing through an intermediate pluripotent stage. Several studies have demonstrated the possibility of switching cell fate by either introducing a single or a combination of reprogramming TFs, inspired by embryonic development or by their in vivo excision (Sancho-Martinez I, et al. Nature Cell Biology. 2012 14 (9): 892-9; Graf T, et al. Nature. 2009 462 (7273): 587-94). The high expression of these TFs aims to overcome current epigenetic marks and push the formation of the target cell. This approach has been explored as an ideal alternative to produce desired cell types, re-shaping traditional concepts of epigenetic stability of differentiated cells, and has been rapidly used to generate functional cells through direct conversion.

The generated cells normally lack mature cell identity and functionality, yet progenitor cells retain a plastic gene expression profile which is correlated with an overall epigenetic remodeling. Progenitors with a fairly open chromatin structure allow to respond to differentiation signals for re-differentiation to a functionally mature state, and the proliferation of those cells permit the generation of plentiful functional cells (Xie B, et al. Cell Research. 2019 29 (9): 696-710). Moreover, the direct change that includes phenotypic changes can be observed from few hours to days after the overexpression of TFs and can progress in the absence of cell proliferation.

There are “pioneer” TFs that act as master regulators of cell fate during normal development, which interact with chromatin and co-TFs (Morris SA. Development. 2016 143 (15): 2696-705). Often, reprogramming strategies use combination of various TFs to overlay the program of the desired cell type by assisting conversion, where one TF can set the fate specification and the other one the maturation, or can alternative act as a repressor to delete the original cellular identity (Ruzittu S, et al. Direct Lineage Reprogramming: Harnessing Cell Plasticity between Liver and Pancreas. Cold Spring Harbor perspectives in biology. 2019). Example TFs for reprogramming cells are provided in Table 1.

TABLE 1
Lineage Reprogramming in human and mice

| | | | |
|---|---|---|---|
| Cell type | Transcription Factors | Target cell type | Description |

Mouse

| | | | |
|---|---|---|---|
| Fibroblasts | Ascl1, Brn2, Myt1l | Glutamatergic | Ascl1 is a master regulator of neural development. |
| | | neurons | The combination of these three factors is sufficient to |
| | | | convert mouse embryonic and postnatal fibroblasts |
| | | | into functional neurons in vitro |
| | Ascl1, Brn2, Myt1l, Lhx3, Hb9, | Motor neuron | Specific differentiated and functional neural subtype |
| | Isl1, Ngn2 | | (spinal motor neuron), can be generated directly from |
| | | | the conversion of mouse fibroblasts |
| | MITF, SOX10, PAX3 | Melanocytes | Combination of these TFs, directly converts mouse |
| | | | fibroblasts to functional melanocytes |
| | Sox2, Brn2, Foxg1 | Neural precursor | Sox2 acts as a pioneer TF. These TFs are able to |
| | | cells | induce neural precursor cells that could be |
| | | | differentiated into neurons, astrocytes, and |
| | | | oligodendrocytes |
| | Nfia, Nfib, Sox9 | Astrocytes | These TFs are identified to be sufficient to convert |
| | | | mouse fibroblasts to astrocytes |
| | Ascl1, Foxa2, Brn2, Myt1l, | Dopaminergic | Induced dopaminergic neuronal cells by a |
| | Lmxla, Nurr1 | neurons | combination of these TFs |
| | C/EBPα, PU.1 | Macrophages | C/EBPα is a pioneer TF and act as a modulator of |
| | | | macrophage-specific gene expression, and PU.1 is |
| | | | necessary for the myeloid conversion |
| | Prdm16, C/EBPβ | Adipocytes (Brown | Fully functional brown fat program can be induced by |
| | | fat cells) | the expression of PRDM16 and C/EBPβ in myoblastic |
| | | | precursors |
| | GATA4, Mef2c, Tbx5 | Cardiomyocytes | GATA4 is a well-characterized pioneer factor. Direct |
| | | | conversion into cardiomyocyte-like cells have been |
| | | | achieved by the overexpression of these TFs in vitro, |
| | | | and in vivo |
| | GATA4, Mef2c, Tbx5, HAND2 | Cardiomyocytes | Conversion was also achieved by adding HAND2 |
| | miR-1, 133,208, and 499, JAK | Cardiomyocytes | The use of miRNA-mediated direct conversion to the |
| | inhibitor I | | cardiomyocyte lineage in vitro and in vivo by using |
| | | | this combination |
| | Oct4, Sox2, Klf4 | Angioblast | These three acts as pioneer TFs. Whit this |
| | | | combination, angioblasts can be obtained |
| | Oct4, Runx2, Osterix, L-Myc | Osteoblasts | Oct4 is a pioneer TF, and the combination have been |
| | | | reported to convert fibroblasts into osteoblasts |
| | Pax7, Mef2b and MyoD | Skeletal muscle | The three TFs can give rise to skeletal muscle |
| | | progenitor cells | progenitor cells, being MyoD the pioneer TF |
| | Sox9, Klf4, c-Myc | Chondrocytes | The conversion is mediated by the expression of two |
| | | | reprogramming factors (c-Myc and Klf4) and one |
| | | | chondrogenic factor (SOX9) |
| | GATA4, FoxA, Hnf4α, Hnf1α | Hepatocytes | Hepatocyte-like cells can mature to functional |
| | | | hepatocytes in vivo by the pioneer TFs FoxA and |
| | | | Hnf4α |
| | Sox10, EGR2 | Schwann cells | Conversion into induced Schwann cells by driving the |
| | | | expression of Sox10 and Egr2 TFs |
| | Etv2, Foxc2, fli1 | Endothelial cells | Induced endothelial cells can be converted from |
| | | | somatic cells in vitro and in vivo |
| B cells | C/EBPα or C/EBPβ, PU.1 | Macrophages | C/EBPα is a pioneer TF, and the overexpression of |
| | | | these TFs induce the transdifferentiation into |
| | | | functional macrophages |
| | Pax5 knockout | T cells | Deletion of Pax5 can induce conversion by the de- |
| | | | differentiation towards a common lymphoid progenitor |
| Alpha cells | Pdx1, MafA, Ngn3 | Beta cells | Conversion into cells that closely resemble endocrine |
| | | | insulin-positive beta cells using a specific combination |
| | | | of these TFs. Mafa potentiates Pdx1 to convert cells |
| | | | to insulin-positive cells |
| Intestinal cells | Pdx1, MafA, Ngn3 | Beta cells | Reprogramming toward a functional β-like cells in vivo |
| Hepatocytes | Pdx1, MafA, Ngn3 | Insulin-producing cell | Reprogramming toward a functional β-like cells in vivo |
| Hepatocytes | Tgif2 | Pancreatic cells | Hepatocytes expressing Tgif2 acquire a pancreatic |
| | | | progenitor state and undergo differentiation in vitro or |
| | | | in vivo |
| Hepatocytes | Ascl1, Brn2, Myt1l | Neurons | Albumin-expressing hepatocytes can be converted |
| | | | into functional neuronal (glutamatergic) |

Human

| | | | |
|---|---|---|---|
| Fibroblasts | Ascl1, Brn2, Myt1l, NeurdoD1 | Glutamatergic | Forced expression of the combination of these TFs, |
| | | neurons | with Ascl1 as pioneer, can efficiently reprogram into |
| | | | functional induced neuronal cells |
| | Ascl1, Lmxla, Nurr1 | Dopaminergic | Induced dopaminergic neuronal cells by a |
| | | neurons | combination of these TFs |
| | GATA4, HAND2, Myocd, Tbx5, | Cardiomyocytes | Human fibroblasts can be reprogrammed to cardiac- |
| | mIR-1, mIR-133 | | like myocytes by forced expression of these TFs that |
| | | | activate cardiac marker expression |
| | Ascl1, Brn2, Myt1l, Lhx3, Hb9, | Motor neuron | Specific differentiated and functional neural subtype |
| | Isl1, Ngn2 | | (spinal motor neuron), can be generated directly from |
| | | | the conversion of human fibroblasts |
| | Hnf4α, Hnf1α, Hnf6, CEBPA, | Hepatocytes | Functional human induced hepatocytes can be |
| | ATf5, Prox1, p-53-siRNA, C- | | created by overexpressing these TFs |
| | Myc | | |
| | MITF, SOX10, PAX3 | Melanocytes | Combination of these TFs, directly converts human |
| | | | fibroblasts to functional melanocytes |
| | Etv2, Foxc2, fli1 | Endothelial cells | Induced human endothelial cells can be converted |
| | | | from somatic cells in vitro |
| | Oct4 | Hematopoietic | Ectopic expression of this TF can give rise to |
| | | progenitor and | granulocytic, monocytic, megakaryocytic and |
| | | mature cells | erythroid lineages |
| | Sox2, mIR-125b | Monocyte-like | Conversion to hematopoietic progenitor-like cells with |
| | | progenitor cells | macrophage differentiation potential |
| | Sox10, EGR2 | Schwann cells | Conversion into induced Schwann cells by driving the |
| | | | expression of Sox10 and Egr2 TFs |
| | ETS2, MESP1 | Cardiac progenitors | Forced co-expression of these two TFs reprogram |
| | | | fibroblasts into cardiac progenitors |
| | Prdm16, C/EBPβ | Adipocytes (Brown | Fully functional brown fat program can be induced by |
| | | fat cells) | the expression of PRDM16 and C/EBPβ in myoblastic |
| | | | precursors |
| Endothelial cells | Fosb, Gfi1, Runx1, Spi1 | Hematopoietic | These cells can be transduced to induce development |
| | | progenitors | of hematopoietic colonies comprising cells with |
| | | | functional and phenotypic features of progenitor cells |
| Pancreatic | Activated MAPK and STAT3 | Beta cells | Human exocrine cells can be reprogrammed to |
| exocrine cells | | | insulin-producing cells that acquire functionality |

WO2018119091 describes the use of ETV2, FOXC2, and FLU (“EFF factors”) to reprogram somatic cells into vasculogenic cells, which is incorporated by reference for the teaching of these reprogramming transcription factors. WO2020028697 describes the use of Pdx1, Ng3, Mafa, and Tcf3 (“PMN-T factors”) to reprogram somatic cells into insulin-producing cells, which is incorporated by reference for the teaching of these reprogramming transcription factors. In some embodiments, the transcription factors of the disclosed methods are not EFF OR PMN-T factors.

A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion.

In some embodiments, after transfecting target cells with transcription factors, the cells can then pack the transfected genes (e.g. cDNA) into EVs, which can then induce other skin cells to form insulin-producing cells. Therefore, also disclosed is a method of reprogramming skin cells into insulin-producing cells that involves exposing the somatic cell with an extracellular vesicle produced from a cell containing or Pdx1, Ng3, Mafa, and Tcf3.

EVs secreted by the donor cells can then collected from the culture medium. These EVs can then be administered to the skin cells to reprogram them into insulin-producing cells. In some embodiments, the donor cells can be any cell from the subject able to produce EVs, including (but not limited to) skin cells (e.g., fibroblasts, keratinocytes, skin stem cells), adipocytes, dendritic cells, peripheral blood mononuclear cells (PBMC), pancreatic cells (e.g., ductal epithelial cells), liver cells (e.g., hepatocytes), immune cells (e.g., T cells, macrophages, myeloid derived suppressor cells).

Exosomes and microvesicles are EVs that differ based on their process of biogenesis and biophysical properties, including size and surface protein markers. Exosomes are homogenous small particles ranging from 40 to 150 nm in size and they are normally derived from the endocytic recycling pathway. In endocytosis, endocytic vesicles form at the plasma membrane and fuse to form early endosomes. These mature and become late endosomes where intraluminal vesicles bud off into an intra-vesicular lumen. Instead of fusing with the lysosome, these multivesicular bodies directly fuse with the plasma membrane and release exosomes into the extracellular space. Exosome biogenesis, protein cargo sorting, and release involve the endosomal sorting complex required for transport (ESCRT complex) and other associated proteins such as Alix and Tsg101. In contrast, microvesicles, are produced directly through the outward budding and fission of membrane vesicles from the plasma membrane, and hence, their surface markers are largely dependent on the composition of the membrane of origin. Further, they tend to constitute a larger and more heterogeneous population of extracellular vesicles, ranging from 150 to 1000 nm in diameter. However, both types of vesicles have been shown to deliver functional mRNA, miRNA and proteins to recipient cells.

In some embodiments, the polynucleotides are delivered to the somatic cells, or the donor cells for EVs, intracellularly via a gene gun, a microparticle or nanoparticle suitable for such delivery, transfection by electroporation, three-dimensional nanochannel electroporation, a tissue nanotransfection device, a liposome suitable for such delivery, or a deep-topical tissue nanoelectroinjection device. In some embodiments, a viral vector can be used. However, in other embodiments, the polynucleotides are not delivered virally.

Electroporation is a technique in which an electrical field is applied to cells in order to increase permeability of the cell membrane, allowing cargo (e.g., reprogramming factors) to be introduced into cells. Electroporation is a common technique for introducing foreign DNA into cells.

Tissue nanotransfection allows for direct cytosolic delivery of cargo (e.g., reprogramming factors) into cells by applying a highly intense and focused electric field through arrayed nanochannels, which benignly nanoporates the juxtaposing tissue cell members, and electrophoretically drives cargo into the cells.

In one embodiment, the disclosed compositions are administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1μg to about 50 mg per kg of body weight, from about 1 mg to about 500 mg per kg of body weight; and from about 1 mg to about 50 mg per kg of body weight. Alternatively, the amount of the disclosed compositions administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

In order to express a polypeptide or functional nucleic acid, the nucleotide coding sequence may be inserted into appropriate expression vector. Therefore, also disclosed is a non-viral vector comprising a polynucleotide comprising nucleic acid sequences encoding 2, 3, or 4 of the [symbol] [symbol] , wherein the nucleic acid sequences are operably linked to an expression control sequence. In some embodiments, the nucleic acid sequences are operably linked to a single expression control sequence. In other embodiments, the nucleic acid sequences are operably linked to two or more separate expression control sequences. Methods to construct expression vectors containing genetic sequences and appropriate transcriptional and translational control elements are well known in the art. These methods include in vitro recombinant DNA techniques, synthetic techniques, and in vivo genetic recombination. Such techniques are described in Sambrook et al., Molecular Cloning, A Laboratory Manual (Cold Spring Harbor Press, Plainview, N. Y., 1989), and Ausubel et al., Current Protocols in Molecular Biology (John Wiley & Sons, New York, N.Y., 1989).

Expression vectors generally contain regulatory sequences necessary elements for the translation and/or transcription of the inserted coding sequence. For example, the coding sequence is preferably operably linked to a promoter and/or enhancer to help control the expression of the desired gene product.

The “control elements” or “regulatory sequences” are those non-translated regions of the vector—enhancers, promoters, 5′ and 3′ untranslated regions—which interact with host cellular proteins to carry out transcription and translation. Such elements may vary in their strength and specificity.

A “promoter” is generally a sequence or sequences of DNA that function when in a relatively fixed location in regard to the transcription start site. A “promoter” contains core elements required for basic interaction of RNA polymerase and transcription factors and can contain upstream elements and response elements.

“Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers, like promoters, also often contain response elements that mediate the regulation of transcription. Enhancers often determine the regulation of expression.

An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

Promoters used in biotechnology are of different types according to the intended type of control of gene expression. They can be generally divided into constitutive promoters, tissue-specific or development-stage-specific promoters, inducible promoters, and synthetic promoters.

Constitutive promoters direct expression in virtually all tissues and are largely, if not entirely, independent of environmental and developmental factors. As their expression is normally not conditioned by endogenous factors, constitutive promoters are usually active across species and even across kingdoms. Examples of constitutive promoters include CMV, EF1a, SV40, PGK1, Ubc, Human beta actin, and CAG.

Tissue-specific or development-stage-specific promoters direct the expression of a gene in specific tissue(s) or at certain stages of development. For plants, promoter elements that are expressed or affect the expression of genes in the vascular system, photosynthetic tissues, tubers, roots and other vegetative organs, or seeds and other reproductive organs can be found in heterologous systems (e.g. distantly related species or even other kingdoms) but the most specificity is generally achieved with homologous promoters (i.e. from the same species, genus or family). This is probably because the coordinate expression of transcription factors is necessary for regulation of the promoter's activity.

The performance of inducible promoters is not conditioned to endogenous factors but to environmental conditions and external stimuli that can be artificially controlled. Within this group, there are promoters modulated by abiotic factors such as light, oxygen levels, heat, cold and wounding. Since some of these factors are difficult to control outside an experimental setting, promoters that respond to chemical compounds, not found naturally in the organism of interest, are of particular interest. Along those lines, promoters that respond to antibiotics, copper, alcohol, steroids, and herbicides, among other compounds, have been adapted and refined to allow the induction of gene activity at will and independently of other biotic or abiotic factors.

The two most commonly used inducible expression systems for research of eukaryote cell biology are named Tet-Off and Tet-On. The Tet-Off system makes use of the tetracycline transactivator (ITA) protein, which is created by fusing one protein, TetR (tetracycline repressor), found in Escherichia coli bacteria, with the activation domain of another protein, VP16, found in the Herpes Simplex Virus. The resulting tTA protein is able to bind to DNA at specific TetO operator sequences. In most Tet-Off systems, several repeats of such TetO sequences are placed upstream of a minimal promoter such as the CMV promoter. The entirety of several TetO sequences with a minimal promoter is called a tetracycline response element (TRE), because it responds to binding of the tetracycline transactivator protein tTA by increased expression of the gene or genes downstream of its promoter. In a Tet-Off system, expression of TRE-controlled genes can be repressed by tetracycline and its derivatives. They bind tTA and render it incapable of binding to TRE sequences, thereby preventing transactivation of TRE-controlled genes. A Tet-On system works similarly, but in the opposite fashion. While in a Tet-Off system, tTA is capable of binding the operator only if not bound to tetracycline or one of its derivatives, such as doxycycline, in a Tet-On system, the rtTA protein is capable of binding the operator only if bound by a tetracycline. Thus the introduction of doxycycline to the system initiates the transcription of the genetic product. The Tet-On system is sometimes preferred over Tet-Off for its faster responsiveness.

In some embodiments, the nucleic acid sequences encoding the transcripotion factors are operably linked to the same expression control sequence. Alternatively, internal ribosome entry sites (IRES) elements can be used to create multigene, or polycistronic, messages. IRES elements are able to bypass the ribosome scanning model of 5′ methylated Cap dependent translation and begin translation at internal sites. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Disclosed are non-viral vectors containing one or more polynucleotides disclosed herein operably linked to an expression control sequence. Examples of such non-viral vectors include the oligonucleotide alone or in combination with a suitable protein, polysaccharide or lipid formulation. Non-viral methods present certain advantages over viral methods, with simple large scale production and low host immunogenicity being just two. Previously, low levels of transfection and expression of the gene held non-viral methods at a disadvantage; however, recent advances in vector technology have yielded molecules and techniques with transfection efficiencies similar to those of viruses.

Examples of suitable non-viral vectors include, but are not limited to pIRES-hrGFP-2a, pCMV6, pMAX, pCAG, pAd-IRES-GFP, and pCDNA3.0

The compositions disclosed can be used therapeutically in combination with a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2: 447-451, (1991); Bagshawe, K. D., Br. J. Cancer, 60: 275-281, (1989); Bagshawe, et al., Br. J. Cancer, 58: 700-703, (1988); Senter, et al., Bioconjugate Chem., 4: 3-9, (1993); Battelli, et al., Cancer Immunol. Immunother., 35: 421-425, (1992); Pietersz and Mckenzie, Immunolog. Reviews, 129: 57-80, (1992); and Roffler, et al., Biochem. Pharmacol, 42: 2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49: 6214-6220, (1989); and Litzinger and Huang, Biochimica et Biophysica Acta, 1104: 179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10: 6, 399-409 (1991)).

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid-or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The herein disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

### Engineering Reprogrammed Cells to Produce EVs

These EVs can in some embodiments be used to deliver diagnostic and/or therapeutic cargo to the target cells in a subject in need thereof. Therefore, also disclosed herein are methods of treating a disease or condition in a subject that involves engineering the reprogrammed cells to produce therapeutic EVs that deliver a therapeutic agent to the target cells.

The disclosed EVs can in some embodiments be any vesicle that can be secreted by a cell. Cells secrete extracellular vesicles (EVs) with a broad range of diameters and functions, including apoptotic bodies (1-5 μm), microvesicles (100-1000 nm in size), and vesicles of endosomal origin, known as exosomes (50-150 nm).

The disclosed extracellular vesicles may be prepared by methods known in the art. For example, the disclosed extracellular vesicles may be prepared by expressing in a eukaryotic cell an mRNA that encodes the cell-targeting ligand. In some embodiments, the cell also expresses an mRNA that encodes a therapeutic cargo. The mRNA for the cell-targeting ligand and the therapeutic cargo may be expressed from vectors that are transfected into suitable production cells for producing the disclosed EVs. The mRNA for the cell-targeting ligand and the therapeutic cargo may be expressed from the same vector (e.g., where the vector expresses the mRNA for the cell-targeting ligand and the therapeutic cargo from separate promoters), or the mRNA for the cell-targeting ligand and the therapeutic cargo may be expressed from separate vectors. The vector or vectors for expressing the mRNA for the cell-targeting ligand and the therapeutic cargo may be packaged in a kit designed for preparing the disclosed extracellular vesicles.

### Therapeutic Cargo

The disclosed extracellular vesicles may be loaded with a therapeutic agent, where the extracellular vesicles deliver the agent to target cells. Suitable therapeutic agents include but are not limited to therapeutic drugs (e.g., small molecule drugs), therapeutic proteins, and therapeutic nucleic acids (e.g., therapeutic RNA). In some embodiments, the disclosed extracellular vesicles comprise a therapeutic RNA (also referred to herein as a “cargo RNA”).

For example, in some embodiments a cell-targeting protein also includes an

RNA-domain (e.g., at a cytosolic C-terminus of the fusion protein) that binds to one or more RNA-motifs present in the cargo RNA in order to package the cargo RNA into the extracellular vesicle, prior to the extracellular vesicles being secreted from a cell. As such, the protein may function as both of a “cell-targeting protein” and a “packaging protein.” In some embodiments, the packaging protein may be referred to as extracellular vesicle-loading protein or “EV-loading protein.”

Representative examples of nucleic acid therapeutic, prophylactic and diagnostic agents include DNA plasmid vectors such as expression vectors, RNA molecules such as iRNA, siRNA, ribozymes, aptamers, repRNAs, gRNA/sgRNA (guide RNA/single guide RNA for CRISPR-based gene editing), and mRNAs.

The cargo RNA of the disclosed extracellular vesicles may be of any suitable length. For example, in some embodiments the cargo RNA may have a nucleotide length of at least about 10 nt, 20 nt, 30 nt, 40 nt, 50 nt, 100 nt, 200 nt, 500 nt, 1000 nt, 2000 nt, 5000 nt, or longer. In other embodiments, the cargo RNA may have a nucleotide length of no more than about 5000 nt, 2000 nt, 1000 nt, 500 nt, 200 nt, 100 nt, 50 nt, 40 nt, 30 nt, 20 nt, or 10 nt. In even further embodiments, the cargo RNA may have a nucleotide length within a range of these contemplated nucleotide lengths, for example, a nucleotide length between a range of about 10 nt-5000 nt, or other ranges. The cargo RNA of the disclosed extracellular vesicles may be relatively long, for example, where the cargo RNA comprises an mRNA or another relatively long RNA.

In some embodiments, the therapeutic cargo is a membrane-permeable pharmacological compound that is loaded into the EV after it is secreted by the cell.

To achieve loading of small RNAs into EVs, transfection-based approaches have been proposed. Other reports have shown that using vector-induced expression of small RNAs in cells, small RNA loading into EVs can be achieved. Alternatively, EV donor cells may be transfected with small RNAs directly. Incubation of tumor cells with chemotherapeutic drugs is also another method to package drugs into EVs. To stimulate formation of drug-loaded EVs, cells are irradiated with ultraviolet light to induce apoptosis. Alternative approaches such as fusogenic liposomes also leads loading drugs into EVs.

In some embodiments, the therapeutic cargo is loaded into the EVs by diffusion via a concentration gradient.

gRNA

In some embodiments, the therapeutic RNA is a guide RNA (gRNA). The guide RNA may guide a Cas nuclease to a target sequence on a target nucleic acid molecule, where the guide RNA hybridizes with and the Cas nuclease cleaves or modulates the target sequence. In some embodiments, a guide RNA binds with and provides specificity of cleavage by a nuclease. In some embodiments, the guide RNA and the Cas protein may form a ribonucleoprotein (RNP), e.g., a CRISPR/Cas complex. In some embodiments, the CRISPR complex may be a Type-II CRISPR/Cas9 complex. In some embodiments, the CRISPR/Cas complex may be a Type-V CRISPR/Cas complex, such as a Cpf1/guide RNA complex. In some embodiments, the Cas nuclease may be a single-protein Cas nuclease, e.g. a Cas9 protein or a Cpf1 protein. In some embodiments, the guide RNA targets cleavage by a Cas9 protein.

In some embodiments, a guide RNA for a CRISPR/Cas9 nuclease system comprises a CRISPR RNA (crRNA) and a tracr RNA (tracr). In some embodiments, the crRNA may comprise a targeting sequence that is complementary to and hybridizes with the target sequence on the target nucleic acid molecule. The crRNA may also comprise a flagpole that is complementary to and hybridizes with a portion of the tracrRNA. In some embodiments, the crRNA may parallel the structure of a naturally occurring crRNA transcribed from a CRISPR locus of a bacteria, where the targeting sequence acts as the spacer of the CRISPR/Cas9 system, and the flagpole corresponds to a portion of a repeat sequence flanking the spacers on the CRISPR locus.

The guide RNA may target any sequence of interest via the targeting sequence of the crRNA. In some embodiments, the degree of complementarity between the targeting sequence of the guide RNA and the target sequence on the target nucleic acid molecule may be about 60%, 65%, 70%, 75%, 80%, 85%, 90%, 95%, 97%, 98%, 99%, or 100%. In some embodiments, the targeting sequence of the guide RNA and the target sequence on the target nucleic acid molecule may be 100% complementary. In other embodiments, the targeting sequence of the guide RNA and the target sequence on the target nucleic acid molecule may contain at least one mismatch. For example, the targeting sequence of the guide RNA and the target sequence on the target nucleic acid molecule may contain 1, 2, 3, 4, 5, 6, 7, 8, 9, or 10 mismatches. In some embodiments, the targeting sequence of the guide RNA and the target sequence on the target nucleic acid molecule may contain 1-6 mismatches. In some embodiments, the targeting sequence of the guide RNA and the target sequence on the target nucleic acid molecule may contain 5 or 6 mismatches.

The length of the targeting sequence may depend on the CRISPR/Cas system and components used. For example, different Cas proteins from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence may comprise 18-24 nucleotides in length. In some embodiments, the targeting sequence may comprise 19-21 nucleotides in length. In some embodiments, the targeting sequence may comprise 20 nucleotides in length.

The flagpole may comprise any sequence with sufficient complementarity with a tracr RNA to promote the formation of a functional CRISPR/Cas complex. In some embodiments, the flagpole may comprise all or a portion of the sequence (also called a “tag” or “handle”) of a naturally-occurring crRNA that is complementary to the tracr RNA in the same CRISPR/Cas system. In some embodiments, the flagpole may comprise all or a portion of a repeat sequence from a naturally-occurring CRISPR/Cas system. In some embodiments, the flagpole may comprise a truncated or modified tag or handle sequence. In some embodiments, the degree of complementarity between the tracr RNA and the portion of the flagpole that hybridizes with the tracr RNA along the length of the shorter of the two sequences may be about 40%, 50%, 60%, 70%, 80%, or higher, but lower than 100%. In some embodiments, the tracr RNA and the portion of the flagpole that hybridizes with the tracr RNA are not 100% complementary along the length of the shorter of the two sequences because of the presence of one or more bulge structures on the tracr and/or wobble base pairing between the tracr and the flagpole. The length of the flagpole may depend on the CRISPR/Cas system or the tracr RNA used. For example, the flagpole may comprise 10-50 nucleotides, or more than 50 nucleotides in length. In some embodiments, the flagpole may comprise 15-40 nucleotides in length. In other embodiments, the flagpole may comprise 20-30 nucleotides in length. In yet other embodiments, the flagpole may comprise 22 nucleotides in length. When a dual guide RNA is used, for example, the length of the flagpole may have no upper limit.

In some embodiments, the tracr RNA may comprise all or a portion of a wild-type tracr RNA sequence from a naturally-occurring CRISPR/Cas system. In some embodiments, the tracr RNA may comprise a truncated or modified variant of the wild-type tracr RNA. The length of the tracr RNA may depend on the CRISPR/Cas system used. In some embodiments, the tracr RNA may comprise 5, 6, 7, 8, 9, 10, 11, 12, 13, 14, 15, 16, 17, 18, 19, 20, 25, 30, 40, 50, 60, 70, 80, 90, 100, or more than 100 nucleotides in length. In certain embodiments, the tracr is at least 26 nucleotides in length. In additional embodiments, the tracr is at least 40 nucleotides in length. In some embodiments, the tracr RNA may comprise certain secondary structures, such as, e.g., one or more hairpins or stem-loop structures, or one or more bulge structures.

In some embodiments, the guide RNA may comprise two RNA molecules and is referred to herein as a “dual guide RNA” or “dgRNA”. In some embodiments, the dgRNA may comprise a first RNA molecule comprising a crRNA, and a second RNA molecule comprising a tracr RNA. The first and second RNA molecules may form a RNA duplex via the base pairing between the flagpole on the crRNA and the tracr RNA.

In some embodiments, the guide RNA may comprise a single RNA molecule and is referred to herein as a “single guide RNA” or “sgRNA”. In some embodiments, the sgRNA may comprise a crRNA covalently linked to a tracr RNA. In some embodiments, the crRNA and the tracr RNA may be covalently linked via a linker. In some embodiments, the single-molecule guide RNA may comprise a stem-loop structure via the base pairing between the flagpole on the crRNA and the tracr RNA. In some embodiments, the sgRNA is a “Cas9 sgRNA” capable of mediating RNA-guided DNA cleavage by a Cas9 protein. In some embodiments, the sgRNA is a “Cpf1 sgRNA” capable of mediating RNA-guided DNA cleavage by a Cpf1 protein. In some embodiments, the guide RNA comprises a crRNA and tracr RNA sufficient for forming an active complex with a Cas9 protein and mediating RNA-guided DNA cleavage. In some embodiments, the guide RNA comprises a crRNA sufficient for forming an active complex with a Cpf1 protein and mediating RNA-guided DNA cleavage.

In some embodiments, the cargo involves expression cassettes encoding a guide RNA. A “guide RNA nucleic acid” can therefore refer to a guide RNA (e.g. an sgRNA or a dgRNA) and a guide RNA expression cassette, which is a nucleic acid that encodes one or more guide RNAs.

In some embodiments, the nucleic acid may be a DNA molecule. In some embodiments, the nucleic acid may comprise a nucleotide sequence encoding a crRNA.

In some embodiments, the nucleotide sequence encoding the crRNA comprises a targeting sequence flanked by all or a portion of a repeat sequence from a naturally-occurring CRISPR/Cas system. In some embodiments, the nucleic acid may comprise a nucleotide sequence encoding a tracr RNA. In some embodiments, the crRNA and the tracr RNA may be encoded by two separate nucleic acids. In other embodiments, the crRNA and the tracr RNA may be encoded by a single nucleic acid. In some embodiments, the crRNA and the tracr RNA may be encoded by opposite strands of a single nucleic acid. In other embodiments, the crRNA and the tracr RNA may be encoded by the same strand of a single nucleic acid. In some embodiments, the expression cassette encodes an sgRNA. In some embodiments, the expression cassette encodes a Cas9 nuclease sgRNA. In come embodiments, the expression cassette encodes a Cpf1 nuclease sgRNA.

The nucleotide sequence encoding the guide RNA may be operably linked to at least one transcriptional or regulatory control sequence, such as a promoter, a 3′ UTR, or a 5′ UTR. In one example, the promoter may be a tRNA promoter, e.g., tRNA^{Lys3}, or a tRNA chimera. See Mefferd et al., RNA. 2015 21: 1683-9; Scherer et al., Nucleic Acids Res. 2007 35: 2620-2628. In certain embodiments, the promoter may be recognized by RNA polymerase III (Pol III). Non-limiting examples of Pol III promoters also include U6 and H1 promoters. In some embodiments, the nucleotide sequence encoding the guide RNA may be operably linked to a mouse or human U6 promoter. In some embodiments, the expression cassette is a modified nucleic acid. In certain embodiments, the expression cassette includes a modified nucleoside or nucleotide. In some embodiments, the expression cassette includes a 5′ end modification, for example a modified nucleoside or nucleotide to stabilize and prevent integration of the expression cassette. In some embodiments, the expression cassette comprises a double-stranded DNA having a 5′ end modification on each strand. In certain embodiments, the expression cassette includes an inverted dideoxy-T or an inverted abasic nucleoside or nucleotide as the 5′ end modification. In some embodiments, the expression cassette includes a label such as biotin, desthiobioten-TEG, digoxigenin, and fluorescent markers, including, for example, FAM, ROX, TAMRA, and AlexaFluor.

In certain embodiments, more than one guide RNA can be used with a CRISPR/Cas nuclease system. Each guide RNA may contain a different targeting sequence, such that the CRISPR/Cas system cleaves more than one target sequence. In some embodiments, one or more guide RNAs may have the same or differing properties such as activity or stability within a CRISPR/Cas complex. Where more than one guide RNA is used, each guide RNA can be encoded on the same or on different expression cassettes. The promoters used to drive expression of the more than one guide RNA may be the same or different.

### Template Nucleic Acid

The formulations disclosed herein may include a template nucleic acid. The template may be used to alter or insert a nucleic acid sequence at or near a target site for a Cas nuclease.

In some embodiments, the template may be used in homologous recombination. In some embodiments, the homologous recombination may result in the integration of the template sequence or a portion of the template sequence into the target nucleic acid molecule. In some embodiments, a single template may be provided. In other embodiments, two or more templates may be provided such that homologous recombination may occur at two or more target sites. For example, different templates may be provided to repair a single gene in a cell, or two different genes in a cell. In some embodiments, multiple copies of at least one template are provided to a cell. In some embodiments, the different templates may be provided in independent copy numbers or independent amounts.

In other embodiments, the template may be used in homology-directed repair, which involves DNA strand invasion at the site of the cleavage in the nucleic acid. In some embodiments, the homology-directed repair may result in including the template sequence in the edited target nucleic acid molecule. In some embodiments, a single template may be provided. In other embodiments, two or more templates having different sequences may be used at two or more sites by homology-directed repair. For example, different templates may be provided to repair a single gene in a cell, or two different genes in a cell. In some embodiments, multiple copies of at least one template are provided to a cell. In some embodiments, the different templates may be provided in independent copy numbers or independent amounts.

In yet other embodiments, the template may be used in gene editing mediated by non-homologous end joining. In some embodiments, the template sequence has no similarity to the nucleic acid sequence near the cleavage site. In some embodiments, the template or a portion of the template sequence is incorporated. In some embodiments, a single template may be provided. In other embodiments, two or more templates having different sequences may be inserted at two or more sites by non-homologous end joining. For example, different templates may be provided to insert a single template in a cell, or two different templates in a cell. In some embodiments, the different templates may be provided in independent copy numbers. In some embodiments, the template includes flanking inverted terminal repeat (ITR) sequences.

In some embodiments, the template sequence may correspond to an endogenous sequence of a target cell. As used herein, the term “endogenous sequence” refers to a sequence that is native to the cell. The term “exogenous sequence” refers to a sequence that is not native to a cell, or a sequence whose native location in the genome of the cell is in a different location. In some embodiments, the endogenous sequence may be a genomic sequence of the cell. In some embodiments, the endogenous sequence may be a chromosomal or extrachromosomal sequence. In some embodiments, the endogenous sequence may be a plasmid sequence of the cell. In some embodiments, the template sequence may be substantially identical to a portion of the endogenous sequence in a cell at or near the cleavage site, but comprise at least one nucleotide change. In some embodiments, the repair of the cleaved target nucleic acid molecule with the template may result in a mutation comprising an insertion, deletion, or substitution of one or more nucleotides of the target nucleic acid molecule. In some embodiments, the mutation may result in one or more amino acid changes in a protein expressed from a gene comprising the target sequence. In some embodiments, the mutation may result in one or more nucleotide changes in an RNA expressed from the target gene. In some embodiments, the mutation may alter the expression level of the target gene. In some embodiments, the mutation may result in increased or decreased expression of the target gene. In some embodiments, the mutation may result in gene knockdown. In some embodiments, the mutation may result in gene knockout. In some embodiments, the mutation may result in restored gene function. In some embodiments, the repair of the cleaved target nucleic acid molecule with the template may result in a change in an exon sequence, an intron sequence, a regulatory sequence, a transcriptional control sequence, a translational control sequence, a splicing site, or a non-coding sequence of the target gene.

In other embodiments, the template sequence may comprise an exogenous sequence. In some embodiments, the exogenous sequence may comprise a protein or RNA coding sequence operably linked to an exogenous promoter sequence such that, upon integration of the exogenous sequence into the target nucleic acid molecule, the cell is capable of expressing the protein or RNA encoded by the integrated sequence. In other embodiments, upon integration of the exogenous sequence into the target nucleic acid molecule, the expression of the integrated sequence may be regulated by an endogenous promoter sequence. In some embodiments, the exogenous sequence may be a chromosomal or extrachromosomal sequence. In some embodiments, the exogenous sequence may provide a cDNA sequence encoding a protein or a portion of the protein. In yet other embodiments, the exogenous sequence may comprise an exon sequence, an intron sequence, a regulatory sequence, a transcriptional control sequence, a translational control sequence, a splicing site, or a non-coding sequence. In some embodiments, the integration of the exogenous sequence may result in restored gene function. In some embodiments, the integration of the exogenous sequence may result in a gene knock-in. In some embodiments, the integration of the exogenous sequence may result in a gene knock-out.

The template may be of any suitable length. In some embodiments, the template may comprise 10, 15, 20, 25, 50, 75, 100, 150, 200, 500, 1000, 1500, 2000, 2500, 3000, 3500, 4000, 4500, 5000, 5500, 6000, or more nucleotides in length. The template may be a single-stranded nucleic acid. The template can be double-stranded or partially double-stranded nucleic acid. In certain embodiments, the single stranded template is 20, 30, 40, 50, 75, 100, 125, 150, 175, or 200 nucleotides in length. In some embodiments, the template may comprise a nucleotide sequence that is complementary to a portion of the target nucleic acid molecule comprising the target sequence (i.e., a “homology arm”). In some embodiments, the template may comprise a homology arm that is complementary to the sequence located upstream or downstream of the cleavage site on the target nucleic acid molecule. In some embodiments, the template may comprise a first homology arm and a second homology arm (also called a first and second nucleotide sequence) that are complementary to sequences located upstream and downstream of the cleavage site, respectively. Where a template contains two homology arms, each arm can be the same length or different lengths, and the sequence between the homology arms can be substantially similar or identical to the target sequence between the homology arms, or it can be entirely unrelated. In some embodiments, the degree of complementarity between the first nucleotide sequence on the template and the sequence upstream of the cleavage site, and between the second nucleotide sequence on the template and the sequence downstream of the cleavage site, may permit homologous recombination, such as, e.g., high-fidelity homologous recombination, between the template and the target nucleic acid molecule. In some embodiments, the degree of complementarity may be about 50%, 55%, 60%, 65%, 70%, 75%, 80%, 85%, 90%, 95%, 97%, 98%, 99%, or 100%. In some embodiments, the degree of complementarity may be about 95%, 97%, 98%, 99%, or 100%. In some embodiments, the degree of complementarity may be at least 98%, 99%, or 100%. In some embodiments, the degree of complementarity may be 100%.

In some embodiments, the template contains ssDNA or dsDNA containing flanking invert-terminal repeat (ITR) sequences. In some embodiments, the template is supplied as a plasmid, minicircle, nanocircle, or PCR product.

### Chemically Modified RNAs

Modified nucleosides or nucleotides can be present in a therapeutic RNA. The term “modified” RNA to refers to the presence of one or more non-naturally and/or naturally occurring components or configurations that are used instead of or in addition to the canonical A, G, C, and U residues. In some embodiments, a modified RNA is synthesized with a non-canonical nucleoside or nucleotide, here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

The modifications listed above can be combined to provide modified RNAs comprising nucleosides and nucleotides (collectively “residues”) that can have two, three, four, or more modifications. For example, a modified residue can have a modified sugar and a modified nucleobase. In some embodiments, every base of a RNA is modified, e.g., all bases have a modified phosphate group, such as a phosphorothioate group. In certain embodiments, all, or substantially all, of the phosphate groups of an RNA molecule are replaced with phosphorothioate groups. In some embodiments, modified RNAs comprise at least one modified residue at or near the 5′ end of the RNA. In some embodiments, modified RNAs comprise at least one modified residue at or near the 3′ end of the RNA.

In some embodiments, the RNA comprises one, two, three or more modified residues. In some embodiments, the RNA comprises one, two, three or more modified residues at each of the 5′ and the 3′ ends of the RNA. In some embodiments the RNA comprises 5, 10, 15, 50, 100, 200, 300, 400, 500, 600, 700, 800, 900, 1000, or more modified residues. In some embodiments, at least 5% (e.g., at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%) of the positions in a modified RNA are modified nucleosides or nucleotides.

Unmodified nucleic acids can be prone to degradation by, e.g., cellular nucleases. For example, nucleases can hydrolyze nucleic acid phosphodiester bonds. Accordingly, in one aspect the RNAs described herein can contain one or more modified nucleosides or nucleotides, e.g., to introduce stability toward nucleases. In certain embodiments, the cargo RNAs described herein can contain one or more modified nucleosides or nucleotides, e.g., to introduce stability toward nucleases. In some embodiments, the modified RNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂) n-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can included “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative). “Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., —NH₂, alkylamino, dialkylamino, heterocyclyl, arylamino, diarylamino, heteroarylamino, diheteroarylamino, or amino acid); NH(CH₂CH₂NH),CH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

### Pharmaceutical Compositions and Administration

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid-or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The herein disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

### Methods

Disclosed herein are methods for delivering diagnostic or therapeutic cargo to target cells using the disclosed EVs. Therefore, also disclosed herein is a method for treating any disease or condition associated with the target cells.

### Diseases

In some embodiments, the disclosed EVs can be used to treat Neurofibromatosis type 1 (NF1) by delivering NF1. NF1-loaded EVs that selectively target diseased Schwann Cells (SCs) in peripheral nerves via functionalization with SC-targeting ligands, or by reprogramming fibroblasts into SCs to produce EVs with tropism for SCs, and efficiently deliver therapeutic cargo for NF1.

In some embodiments, the disclosed EVs can be used to treat Acute respiratory distress syndrome (ARDS) by delivering anti-inflammatory cargo, such as IL-4, IL-10, FGF7, mIR-146a. Engineered EVs loaded with anti-inflammatory cargo will preferentially target specific cellular components in the alveolar microenvironment in the injured lung, to dampen inflammation and facilitate recovery of patients with ARDS.

In some embodiments, the disclosed EVs can be used to treat Type II diabetes by delivering transcription factors, such as Pdx1, Ngn3, or MafA. Engineered EVs as targeted delivery vehicles of transcription factors to induce cell reprogramming of pancreatic ductal cells into an insulin-producing phenotype.

In some embodiments, the disclosed EVs can be used to treat neurological conditions (neurodegenerative diseases, brain, and nerve injury) by delivering transcription factors, such as Acsl1, Brn2, Myt1l. EVs loaded with reprogramming transcription factors that modulate electrophysiological activity in neuronal cells and drive pro-neurogenic reprogramming in non-neuronal cells, and the potential deployment of functionalized EV-based therapies to the brain. In some embodiments, the disclosed EVs can be used to treat neurological conditions (neurodegenerative diseases, brain, and nerve injury) by delivering dopamine or precursors that would boost dopamine (e.g., dopamine genes such as DRD1, DRD2, DRD3, DRD4, DRD5 and transporter DAT1).

In some embodiments, the disclosed EVs can be used to treat cancer by delivering anti-metastatic and anti-tumor genes, such as Timp3 and Rarres2. Non-viral transfection of myeloid derived suppressor cells (MDSCs) can release engineered EV that can modulate transfer and overexpression of anti-metastatic and anti-tumor genes in tumor cells/tissues in a targeted manner.

In some embodiments, the disclosed EVs can be used to promote wound healing by delivering pro-vascular transcription factors, such as Etv2, Fli1, and Foxc2. EVs loaded with pro-vascular transcription factors induce direct reprogramming of human fibroblasts into endothelial cells, which have the potential to promote healing after injury and develop pre-vascularized skin grafts for regenerative medicine applications.

In some embodiments, the disclosed EVs can be used to treat low back pain by delivering transcription factors such as FOXF1 and/or Brachyury. Engineered EVs can deliver transcription factors that can reprogram degenerate cells in the intervertebral disc (IVD) such as human nucleus pulposus (NP) cells to a healthy pro-anabolic phenotype with increased proteoglycan and decreased inflammatory, catabolic and pain associated factors which are critical for maintaining the structure and function of the healthy IVD.

In some embodiments, the disclosed EVs can be used to treat calcific aortic stenosis by delivering transcription factors such as CEBPa and PU. 1. EV-based therapeutics that selectively deliver reprogramming transcription factors to the affected valve that induce cell reprogramming of endothelial cells towards a pro-healing (M2) macrophage-like phenotype, to promote resorption of the calcified tissue and reduce inflammation.

In some embodiments, the disclosed EVs can be used to treat Alzheimer's disease (AD) by delivering Amyloid beta antagonists such as shRNAs, miRs, siRNAs. Skin derived-EVs has been explored using a model of AD (3×Tgad) in the onset and progression of the disease. These skin derived-EVs are found to be preferentially uptake by neurons and can transfer mRNA to these cells. Increased amyloid beta in hippocampus as well as increased gliosis have been found in AD model, which can be mediated by EVs. Skin derived EVs have been found to also target brain tissue/cells for delivery of cDNA, mRNA, and proteins, which could be of a therapeutic nature. AD progression in the hippocampus has also been found to be partially mediated by EVs, and as such, EVs could potentially be leveraged to deliver therapeutic cargoes to AD-affected cells.

In some embodiments, the disclosed EVs can be used to treat Stroke by delivering pro-vascular transcription factors, such as Etv2, Fli1, and Foxc2. Transfection of fibroblast with transcription factors tend to produce exosomes loaded with pro-vasculogenic/angiogenic transcripts, which suggests that increased intracranial perfusion could be modulated, in part, by exosome-driven autocrine and/or paracrine vasculogenesis and angiogenesis.

In some embodiments, the disclosed EVs can be used to treat peripheral nerve injury by delivering pro-vascular transcription factors, such as Etv2, Fli1, and Foxc2. Delivery of vasculogenic cell therapies to crushed nerves that leads to increased vascularity, reduced macrophage infiltration, and improved recovery in electrophysiological parameters by using reprogramming factor genes via Tissue nanotransfection (TNT).

### Production Method

### Administration

The disclosed EVs may be administered to a subject by any suitable means. Administration to a human or animal subject may be selected from parenteral, intramuscular, intracerebral, intravascular, subcutaneous, or transdermal administration. Typically the method of delivery is by injection. Preferably the injection is intramuscular or intravascular (e.g. intravenous). A physician will be able to determine the required route of administration for each particular patient.

The EVs are preferably delivered as a composition. The composition may be formulated for parenteral, intramuscular, intracerebral, intravascular (including intravenous), subcutaneous, or transdermal administration. Compositions for parenteral administration may include sterile aqueous solutions which may also contain buffers, diluents and other suitable additives. The EVs may be formulated in a pharmaceutical composition, which may include pharmaceutically acceptable carriers, thickeners, diluents, buffers, preservatives, and other pharmaceutically acceptable carriers or excipients and the like in addition to the EVs.

Parenteral administration is generally characterized by injection, such as subcutaneously, intramuscularly, or intravenously. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof. Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include sodium chloride injection, ringers injection, isotonic dextrose injection, sterile water injection, dextrose and lactated ringers injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone.

Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment. The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations can be packaged in an ampoule, a vial or a syringe with a needle. All preparations for parenteral administration should be sterile, as is known and practiced in the art.

A therapeutically effective amount of composition is administered. The dose may be determined according to various parameters, especially according to the severity of the condition, age, and weight of the patient to be treated; the route of administration; and the required regimen. A physician will be able to determine the required route of administration and dosage for any particular patient. Optimum dosages may vary depending on the relative potency of individual constructs, and can generally be estimated based on EC50s found to be effective in vitro and in vivo animal models. In general, dosage is from 0.01 mg/kg to 100 mg per kg of body weight. A typical daily dose is from about 0.1 to 50 mg per kg, preferably from about 0.1 mg/kg to 10 mg/kg of body weight, according to the potency of the specific construct, the age, weight and condition of the subject to be treated, the severity of the disease and the frequency and route of administration. Different dosages of the construct may be administered depending on whether administration is by intramuscular injection or systemic (intravenous or subcutaneous) injection.

Preferably, the dose of a single intramuscular injection is in the range of about 5 to 20 μg. Preferably, the dose of single or multiple systemic injections is in the range of 10 to 100 mg/kg of body weight.

Due to construct clearance (and breakdown of any targeted molecule), the patient may have to be treated repeatedly, for example once or more daily, weekly, monthly or yearly. Persons of ordinary skill in the art can easily estimate repetition rates for dosing based on measured residence times and concentrations of the construct in bodily fluids or tissues. Following successful treatment, it may be desirable to have the patient undergo maintenance therapy, wherein the construct is administered in maintenance doses, ranging from 0.01 mg/kg to 100 mg per kg of body weight, once or more daily, to once every 20 years.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Designer EVs for Targeted Delivery

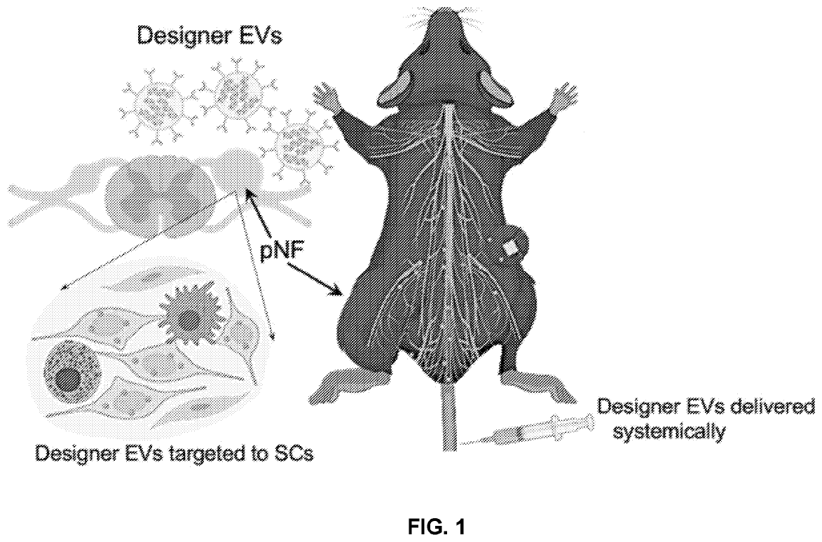

Nanotransfection is used to engineer fibroblasts to produce EVs with tropism for SCs and deliver therapeutic payloads to SCs systemically (FIG. 1).

Neurofibromatosis type 1 (NF1) is driven by mutations/deletions of the NF1 gene in SCs. As NF1 has many key functions (e.g., modulation of Ras-GTPase), such mutations/deletions lead to neurofibromas. As such, gene therapies have been studied to recover NF1 function. Retroviral and adeno-associated viral vectors have been used to transfect the GAP-related domain of NF1 to recover function. However, these studies often yield low efficiencies. Moreover, viral vectors fail to carry full-length NF1 due to capsid size restrictions.

Viral vectors have become the gold standard for gene therapies. However, while promising, viruses have several limitations beyond capsid size constraints. Viral vector-induced immunity, for example, could prevent redosing or pose biosafety concerns. EVs have thus emerged as promising therapeutic carriers for gene therapies. Compared to most carrier systems, viral or synthetic, EVs are able to package large cargos, show improved biocompatibility, reduced immunogenicity, enhanced stability, and an innate ability to pass through biological barriers. As such, a substantial amount of research is being devoted to engineering therapeutic EVs for different diseases. However, currently there is a paucity of research on designer EVs for the delivery of therapeutic payloads for NF1.

EV-based approaches to deploy gene therapies for NF1 are in some embodiments produced by reprogramming fibroblasts into SCs to produce EVs with tropism for SCs.

Full-length NF1 is used as exemplary therapeutic cargo, but the proposed EV technology could be used to deliver different types of therapeutic cargo (e.g., CRISPR/Cas9).

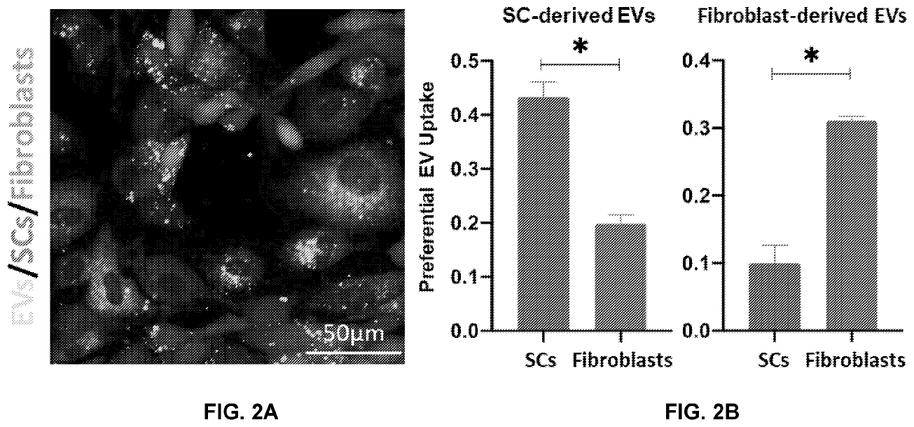

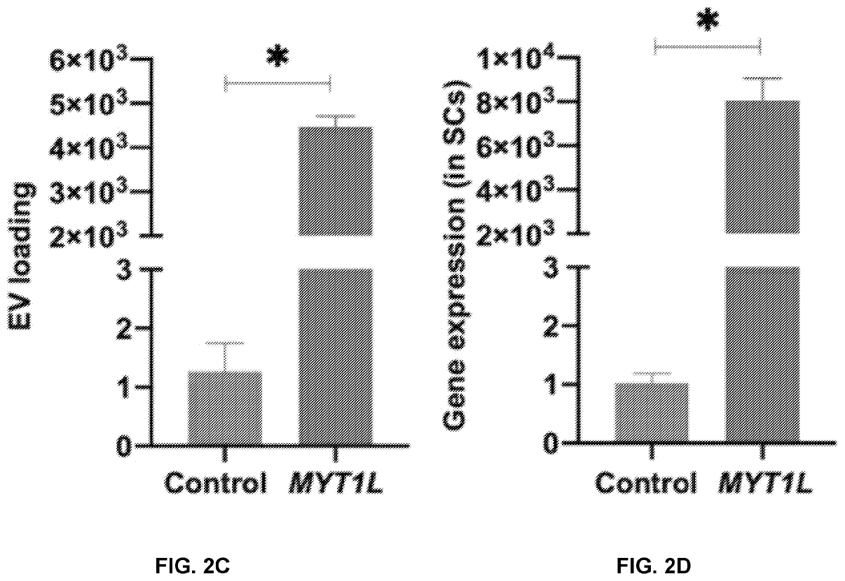

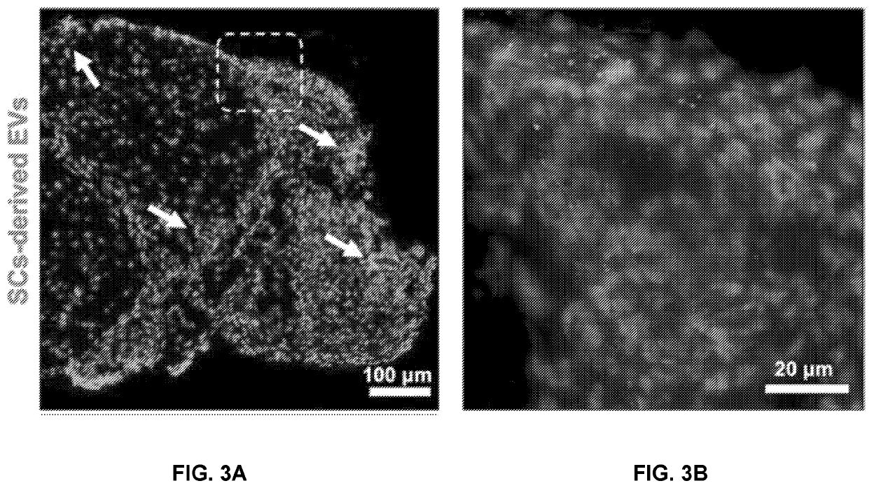

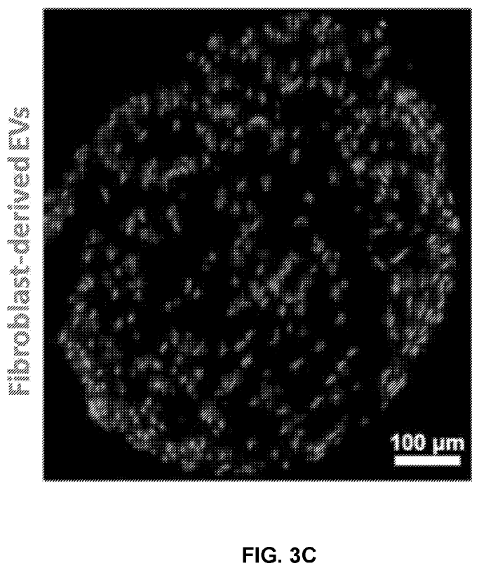

To evaluate whether SC-derived EVs show tropism for SCs, EVs were isolated from nanotransfected SCs (ATCC). Myt1l plasmids (˜10.4 kb) were used as large model cargo. In vitro co-cultures of SCs and fibroblasts exposed to EVs derived from nanotransfected SCs, vs. EVs derived from fibroblasts, show that SC-derived EVs are preferentially captured by SCs compared to fibroblasts, which showed more affinity to fibroblast-derived EVs (FIG. 2A-B). qRTPCR analyses show that the EVs were able to package MYT1L, and mediated gene transfer to SCs (FIG. 2C-D). To test tropism in vivo, a crush injury in the sciatic nerve of mice was performed to induce localized recruitment of SCs. SC-derived EVs were labeled and delivered via tail vein, and the nerve was then collected 6 h later. Imaging of the nerve revealed clear accumulation of SC-derived EVs at the crush site (FIG. 3A-B). Altogether, these data demonstrate the ability to obtain EVs with higher tropism for SCs. Since SCs are not an abundant cell source, methods are developed to impart SC tropism to EVs derived from more readily available cell sources (e.g., dermal fibroblasts).

EVs were isolated from nanotransfected myoblasts with a plasmid encoding for Pmax-GFP To evaluate if myoblast-derived EVs show tropism for myoblasts. In vitro co-cultures of myoblasts and fibroblasts exposed to EVs derived from myoblasts vs. EVs derived from fibroblasts, show that myoblasts-derived EVs are preferentially captured by myoblasts as supposed to fibroblasts, which revealed further affinity to fibroblast-derived EVs (FIG. 42A-B).

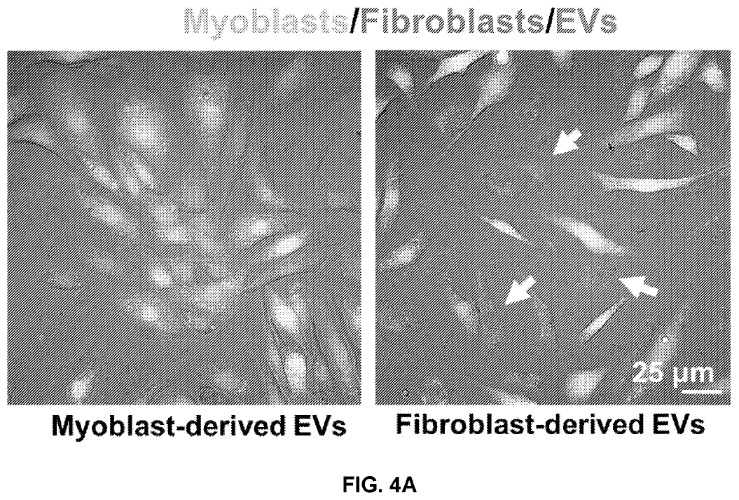

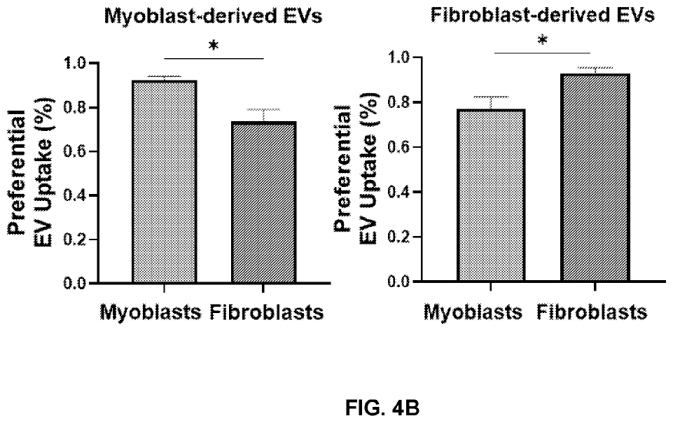

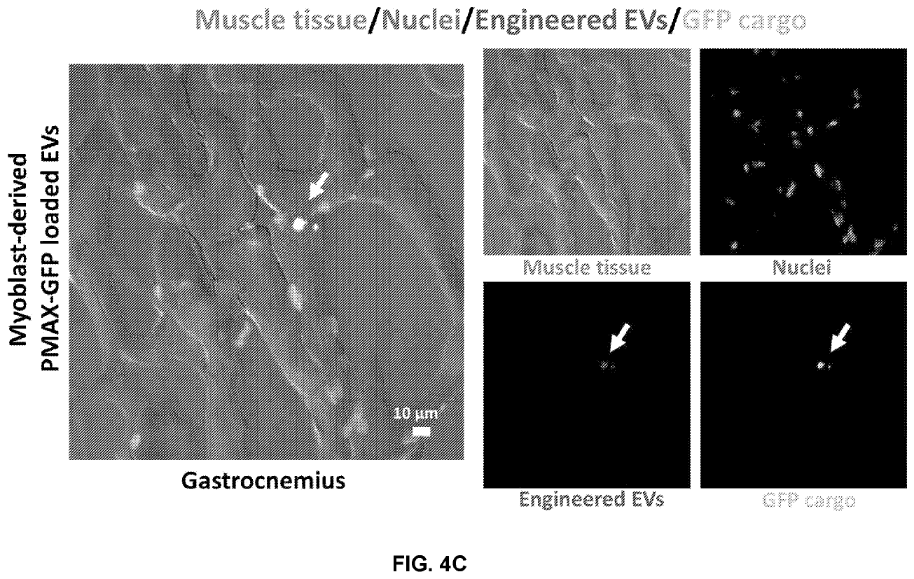

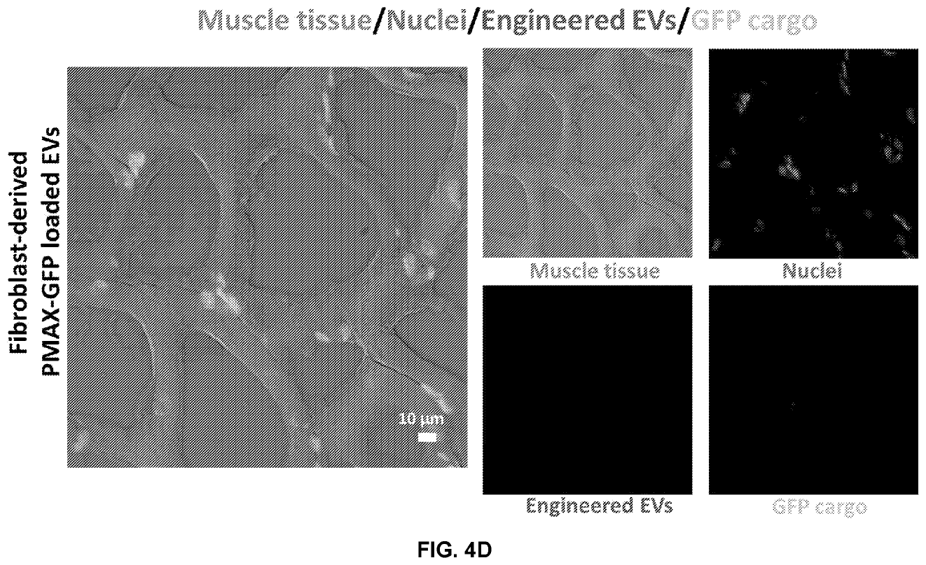

In vivo studies were performed to test tropism of myoblasts derived EVs. For these experiments, myoblasts-derived EVs were labeled and delivered via tail vein, and different muscle tissues (e.g., triceps, quadriceps, abdominals, gastrocnemius) were collected 6 h after. Imaging of the muscle tissue showed accumulation of myoblasts-derived EVs with a co-localization of the labeled and the cargo (GFP) of the EVs with the multiple nuclei of the muscle cells counter stained with DAPI (blue) (FIG. 4C) compared to fibroblast-derived EVs where the signal was absent (FIG. 4D). Altogether, these data demonstrate the ability to obtain EVs with higher tropism for muscle tissue, when the EVs were derived from myoblasts.

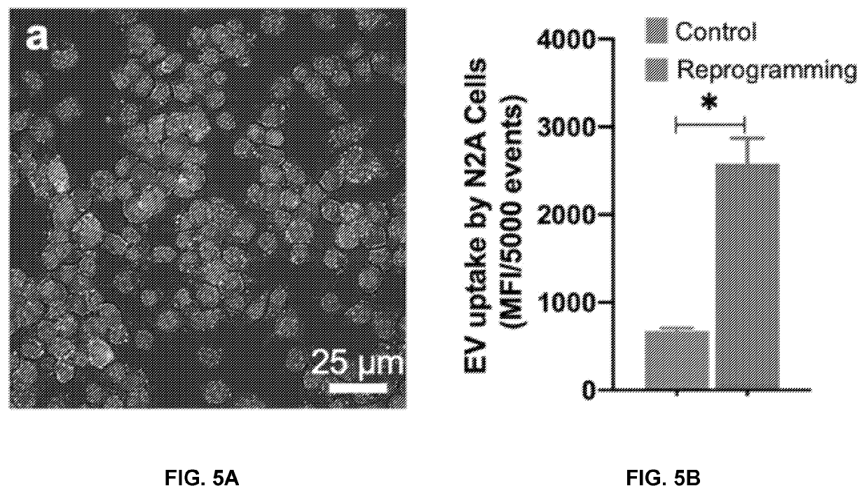

Studies show that nanotransfecting fibroblasts with ASCL1, BRN2, and MYT1L (ABM) leads to reprogramming into neurons. It was evaluated if EVs released during the reprogramming process showed tropism toward neurons (FIG. 5). Uptake studies show that EVs obtained from ABM-nanotransfected fibroblast cells were preferentially internalized by neurons compared to control EVs. These results highlight the ability to use reprogramming to make EVs from fibroblasts with enhanced tropism for other cells.

### Example 2: Develop Designer EVs for SC Targeted Delivery

Designer EV formulations are developed with enhanced tropism for SCs. This is done by nanotransfecting mouse dermal fibroblasts with plasmids to drive conversions into SCs (SOX10, EGR2). The working hypotheses is that EVs from fibroblasts ‘primed’ to convert to SCs show tropism for SCs. SCs are not a readily abundant cell source to generate EVs. Thus, methods are needed to impart SC tropism to EVs derived from more readily abundant cells.

### EVs Derived From Fibroblasts Converting Into SCs

EVs exhibit tropism for cells/tissues from which they originate. Data indicates that SC-derived EVs are preferentially internalized by SCs, and that reprogramming approaches can be leveraged to impart cell-specific tropism to EVs. Here fibroblasts are converted to SCs and EVs are collected at different stages of the reprogramming process. Briefly, fibroblasts are nanotransfected with expression plasmids for SOX10 and EGR219, and EVs are collected from the supernatant at days 1-21. EVs isolated from fibroblasts nanotransfected with sham plasmids serve as controls. SC-directed reprogramming of fibroblasts is evaluated at days 7-21 by qRT-PCR and immunostaining for SC specific markers (S100, O4, and MPZ). NanoSight is used to quantify EV concentration and size.

### Selective Uptake

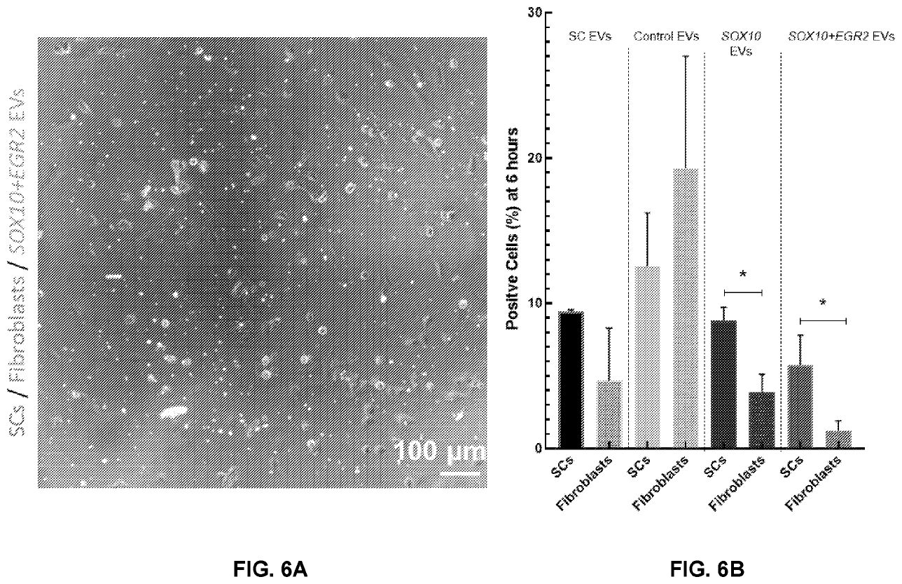

Selective SC uptake of designer EV formulations is assessed in co-cultures of SCs and fibroblasts. SCs (ATCC) and fibroblasts are mixed at a 1:1 ratio. The cells and EVs are labeled with fluorophores of different wavelengths (˜490, 560, and 650 nm). Co-cultures are exposed to ˜10⁹-10¹⁰ EVs/ml, and selective uptake by SCs vs. Fibroblasts are evaluated via confocal microscopy. EVs derived from SCs are used as positive control. Results show that EVs collected 24 hours after transfection of fibroblasts with the reprograming factors SOX10 alone or SOX10+EGR2, are preferentially internalized by SCs compared to fibroblasts 6 hours after EV treatment *p<0.05 (FIG. 6A-B). Biodistribution:

To identify EV formulations with enhanced tropism for SCs in cNFs/pNFs, a murine model of NF1 is used, in which the Nf1 alleles are inactivated in SOX10+ cells, leading to the formation of cNFs and pNFs. Briefly, homozygous Nf1^{flfl} mice (Stock No: 017640, JAX) are mated with tamoxifen-inducible SOX10-CreERT2 mice (Stock No: 027651, JAX). From the offspring, Nf1^{f1/-}:SOX10-CreERT2^{+/0} mice are mated with Nf1^{′flfl} mice. Approximately 25% of the offspring have a homozygous genotype for the Nf1flox allele and hemizygous for the SOX10-CreERT2 allele (Nf1^{flfl}:SOX10-CreERT2⁺⁰) and are used as the experimental line. Offspring homozygous for the Nf1flox allele and null for SOX10-CreERT2 allele (Nf1^{flfl}:SOX10-CreERT2^{0/0}) are used as controls. Mice are treated with tamoxifen at ˜1 month of age. EV formulations are injected via tail vein ˜6 months after tamoxifen induction, once cNF/pNF lesions/symptoms (scruffy fur, hunched posture, limping, limb paralysis) are confirmed. EVs derived from SCs are used as positive control. The EVs are fluorescently tagged with MemGlow. The mice are injected with a bolus of ˜10¹² EVs/gram of weight daily, and mice injected 1-5 times are compared. The mice are euthanized 24 hours after the last injection, and cNF lesions, spinal cord/sciatic nerves (to inspect pNFs), liver, lungs, spleen, and kidneys are collected and imaged by IVIS to evaluate EV distribution. The tissues are subsequently processed for histology. Neurofibromas are immunostained for S100β, GAP43, SOX10, Iba1, and mast cells. The presence of EVs in tissue sections is quantified by confocal imaging.

Delivering NF1 to cNFs/pNFs

Once an optimum timepoint for collection of EVs from fibroblasts primed to convert into SCs is identified, NF1 plasmids are nanotransfected into the converting (or converted) fibroblasts, and EVs are isolated from the supernatant 6-72 h after. Positive and negative control EVs are obtained. NF1 loading is evaluated by qRT-PCR. EVs are delivered to tamoxifen-treated Nf1fl/fl:SOX10-CreERT2^{+/0} mice via tail vein with a bolus of ˜10¹² EVs/gram of weight daily, and mice injected 1-5 times are compared. Biodistribution is assessed. NF1 delivery cNFs/pNFs, and function, are evaluated by qRT-PCR for NF1, and immunostaining for neurofibromin, p-ERK, and quantification of SOX10+ and mast cells. Additional assessments include quantification of the number and volume of neurofibromas, as well as TUNEL and BrdU staining. To verify if EVs carried NF1, laser capture microdissection (LCM) is used to isolate fluorescently tagged portions of tissue sections (indicative of tagged EV accumulation), and PCR/qRT-PCR are used to quantify NF1 plasmid/mRNA at that location.

### EQUIVALENTS AND INCORPORATION BY REFERENCE

All references cited herein are incorporated by reference to the same extent as if each individual publication, database entry (e.g., Genbank sequences or GenelD entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GenelD entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it is understood by persons skilled in the relevant art that various changes in form and details can be made therein without departing from the spirit and scope of the invention.

Unless defined otherwise, all technical and scientific terms used herein have the same meanings as commonly understood by one of skill in the art to which the disclosed invention belongs.

1. A method for producing engineered extracellular vesicles (EVs) with a desired cell tropism for a target cell, the method comprising (a) delivering intracellularly into a population of somatic cells at least one polynucleotide comprising a nucleic acid sequence encoding at least one reprogramming transcription factor configured to reprogram the somatic cells towards the target cell lineage and produce reprogrammed cells; and
(b) collecting the EVs from the reprogrammed cells.

2. The method of claim 1, wherein the somatic cells are selected from the group consisting of a fibroblasts, myoblasts, endothelial cells, and pancreatic exocrine cells.

3. The method of claim 2, wherein the target cell lineage is a glutamatergic neurons, and wherein the reprogramming transcription factors are selected from the group consisting of Ascl1, Brn2, Myt11, and NeurdoD1.

4. The method of claim 2, wherein the target cell lineage is a dopaminergic neuron, and wherein the reprogramming transcription factors are selected from the group consisting of Ascl1, Lmxla, and Nurr1.

5. The method of claim 2, wherein the target cell lineage is a cardiomyocyte, and wherein the reprogramming transcription factors are selected from the group consisting of GATA4, HAND2, Myocd, Tbx5, mIR-1, and mIR-133.

6. The method of claim 2, wherein the target cell lineage is a motor neuron, and wherein the reprogramming transcription factors are selected from the group consisting of Ascl1, Brn2, Myt11, Lhx3, Hb9, Isl1, and Ngn2.

7. (canceled)

8. The method of claim 2, wherein the target cell lineage is a hepatocyte, and wherein the reprogramming transcription factors are selected from the group consisting of Hnf4a, Hnf1a, Hnf6, CEBPA, ATf5, Prox1, p-53-siRNA, and c-Myc.

9. The method of claim 2, wherein the target cell lineage is a melanocyte, and wherein the reprogramming transcription factors are selected from the group consisting of MITF, SOX10, PAX3.

10. The method of claim 2, wherein the target cell lineage is an endothelial cells, and wherein the reprogramming transcription factors are selected from the group consisting of Etv2, Foxc2, and fli1.

11. The method of claim 2, wherein the target cell lineage is a hematopoietic progenitor and mature cell, and wherein the reprogramming transcription factors is Oct4.

12. The method of claim 2, wherein the target cell lineage is a monocyte-like progenitor cell, and wherein the reprogramming transcription factors are selected from the group consisting of Sox2 and mIR-125b.

13. The method of claim 2, wherein the target cell lineage is a Schwann cell, and wherein the reprogramming transcription factors are selected from the group consisting of Sox10 and EGR2.

13. The method of claim 2, wherein the target cell lineage is a cardiac progenitor, and wherein the reprogramming transcription factors are selected from the group consisting of ETS2 and MESP1.

14. The method of claim 2, wherein the target cell lineage is an adipocytes, and wherein the reprogramming transcription factors are selected from the group consisting of Prdm16 and C/EBPβ.

15. (canceled)

16. The method of claim 2, wherein the target cell lineage is a hematopoietic progenitor cell, and wherein the reprogramming transcription factors are selected from the group consisting of Fosb, Gfi1, Runx1, and Spi1.

17. (canceled)

18. The method of claim 2, wherein the target cell lineage is a beta cells, and wherein the reprogramming transcription factors are selected from the group consisting of activated MAPK and STAT3.

19. (canceled)

20. The method of claim 1, wherein the EVs are collected from the reprogrammed cells from 12 hours to 48 hours after the reprogramming transcription factors are delivered to the somatic cells, and wherein the reprogrammed cells express lineage specification markers, but lack one or more markers of full lineage differentiation.

21. (canceled)

22. The method of claim 1, wherein the EVs are decorated with at least one targeting ligand.

23. The method of claim 23, wherein the targeting ligand is a glutamate-targeting ligand.

25. The method of claim 1, further comprising loading the EVs with a therapeutic cargo or engineering the reprogrammed cells to produce a therapeutic cargo that will encapsulate within the EVs.

26. (canceled)
